(19) United States
(12) Patent Application Publication
(10) Pub. No.: US 2015/0132388 A1
(43) Pub. Date: May 14, 2015

(54) COMPLEXES OF FULVESTRANT AND ITS DERIVATIVES, PROCESS FOR THE PREPARATION THEREOF AND PHARMACEUTICAL COMPOSITIONS CONTAINING THEM

(71) Applicant: Druggability Technologies Holdings Limited, Dublin (IE)

(72) Inventors: Erzsébet Réka Angi, Nagykovacsi (HU); Richard Balázs Kárpáti, Tatabanya (HU); Zsolt Ötvös, Csongrad (HU); László Molnár, Biatorbagy (HU); Hristos Glavinas, Szeged (HU); Genovéva Filipcsei, Budapest (HU)

(21) Appl. No.: 14/538,184

(22) Filed: Nov. 11, 2014

(30) Foreign Application Priority Data
Nov. 12, 2013 (HU) P1300646

(51) Int. Cl. A61K 47/48; A61K 9/14; A61K 31/565

(52) U.S. Cl. CPC A61K 47/48215; A61K 31/565; A61K 9/14
USPC 424/489; 514/182; 552/626; 428/402

(57) ABSTRACT

The present invention relates to pharmaceutically acceptable complex formulae comprising complexes of Fulvestrant, or a salt, or derivatives thereof and complexation agents and pharmaceutically acceptable excipients, process for the preparation thereof and pharmaceutical compositions containing them. The complex formulae of the present invention have improved physicochemical properties which makes the compound orally available and makes oral administration of the compound possible in the treatment of hormone receptor positive metastatic breast cancer in postmenopausal women with disease progression following anti-estrogen therapy.

This application claims the benefit of priority to application no. HU P1300646, filed Nov. 12, 2013, the disclosure of which is hereby incorporated by reference as if written herein in its entirety.

### FIELD OF THE INVENTION

The invention is directed to a stable complex with controlled particle size, increased apparent solubility and increased dissolution rate comprising as active compound Fulvestrant, its salts, or derivatives thereof, which is useful in the treatment of hormone receptor positive metastatic breast cancer in postmenopausal women with disease progression following anti-estrogen therapy. More specifically, the complex of the present invention possesses increased apparent solubility, permeability which makes the compound orally available and makes oral administration of the compound possible. The invention also relates to methods of formulating and manufacturing complex according to the invention, pharmaceutical compositions containing it, its uses and methods of treatment using the complex and its compositions.

### BACKGROUND OF THE INVENTION

The chemical name of Fulvestrant is 7-alpha-[9-(4,4,5,5,5-penta fluoropentylsulphinyl)nonyl]estra-1,3,5-(10)-triene-3,17 beta-diol. The molecular formula is C₃₂H₄₇F₅O₃S and its structural formula is:

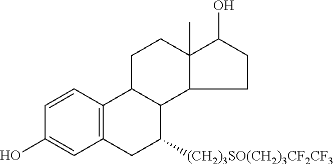

Fulvestrant is a white powder with a molecular weight of 606.77. The solution for injection is a clear, colorless to yellow, viscous liquid.

Each injection contains as inactive ingredients: 10% w/v Alcohol, USP, 10% w/v Benzyl Alcohol, NF, and 15% w/v Benzyl Benzoate, USP, as co-solvents, and made up to 100% w/v with Castor Oil, USP as a co-solvent and release rate modifier.

Many breast cancers have estrogen receptors (ER) and the growth of these tumors can be stimulated by estrogen. Fulvestrant is an estrogen receptor antagonist that binds to the estrogen receptor in a competitive manner with affinity comparable to that of estradiol and downregulates the ER protein in human breast cancer cells.

In vitro studies demonstrated that Fulvestrant is a reversible inhibitor of the growth of tamoxifen-resistant, as well as estrogen-sensitive human breast cancer (MCF-7) cell lines. In in vivo tumor studies, Fulvestrant delayed the establishment of tumors from xenografts of human breast cancer MCF-7 cells in nude mice. Fulvestrant inhibited the growth of established MCF-7 xenografts and of tamoxifen-resistant breast tumor xenografts.

Fulvestrant showed no agonist-type effects in in vivo uterotropic assays in immature or ovariectomized mice and rats. In in vivo studies in immature rats and ovariectomized monkeys, Fulvestrant blocked the uterotrophic action of estradiol. In postmenopausal women, the absence of changes in plasma concentrations of FSH and LH in response to Fulvestrant treatment (250 mg monthly) suggests no peripheral steroidal effects.

After administration of Fulvestrant 250 mg intramuscularly, Fulvestrant is slowly absorbed. Maximum plasma concentrations are reached after about 7 days. Single dose studies have demonstrated that absorption continues for more than one month and that the terminal half-life is about 50 days. The variability in exposure after the first IM LA dose is large; CV is 25-70% for AUC_{0-28d} and 28-83% for Cₘₐₓ. Once a month administration results in approximately 2-3 fold accumulation. Steady state is reached after about 6 months but the majority of the accumulation is achieved after 3-4 doses. At steady state, the Cₘₐₓ/Cₘᵢₙ ratio is ˜2. Considerably lower variability is observed at steady state with CV being ˜15%.

The bioavailability has been estimated to be about 90-100% using between study comparisons. Exposure is approximately proportional to dose in the studied range 50 to 500 mg.

Biotransformation and disposition of Fulvestrant in humans have been determined following intramuscular and intravenous administration of 14C-labeled Fulvestrant. Metabolism of Fulvestrant appears to involve combinations of a number of possible biotransformation pathways analogous to those of endogenous steroids, including oxidation, aromatic hydroxylation, conjugation with glucuronic acid and/or sulphate at the 2, 3 and 17 positions of the steroid nucleus, and oxidation of the side chain sulphoxide. Identified metabolites are either less active or exhibit similar activity to Fulvestrant in antiestrogen models.

Studies using human liver preparations and recombinant human enzymes indicate that cytochrome P-450 3A4 (CYP 3A4) is the only P-450 isoenzyme involved in the oxidation of fulvestrant; however, the relative contribution of P-450 and non-P-450 routes in vivo is unknown.

Fulvestrant was rapidly cleared by the hepatobiliary route with excretion primarily via the feces (approximately 90%). Renal elimination was negligible (less than 1%). After an intramuscular injection of 250 mg, the clearance (Mean±SD) was 690±226 mL/min with an apparent half-life about 40 days.

Fulvestrant could not achieve adequate oral bioavailability due to poor solubility. Fulvestrant has therefore been developed for administration by intramuscular injection. The goal of the development of Fulvestrant intramuscular injection was to achieve effective delivery of active ingredient, using the formulation to control the rate of drug input and reduce the frequency of administration. Studies were carried out to measure Fulvestrant solubility in a range of oils, esters and alcohols suitable for inclusion in intramuscular injection formulations. It was found that castor oil together with co-solvents (benzyl alcohol, ethanol and benzyl benzoate) were the most suitable to allow a Fulvestrant concentration of 50 mg/ml.

The main safety concerns surrounding Fulvestrant injection are related to its intramuscular route of administration. It needs to be used with caution in patients with bleeding disorders, decreased platelet count, or in patients receiving anticoagulants (for example, warfarin), in addition it is associated with injection site pain. A non-intramuscular route of administration would avoid all of these concerns.

Further problem with the current formulation is the requirement to administer two 5 mL injections. The pain associated with these injections is problematic.

The current method of intramuscular administration is limited by the volume (5 mL) of each injection which itself is limited by the solubility of Fulvestrant in castor oil and co-solvents. A novel complex form of Fulvestrant with greater apparent solubility allows much smaller injection volumes, perhaps allowing a reduction in the number of injections from 2 to 1.

Shortening the time to Cₘₐₓ should in theory allow more patients to reach therapeutic concentrations sooner which may result in an improved response rate. Similarly, a reduction in the variability of Cₘₐₓ and AUC may also produce better efficacy.

In order to overcome the problems associated with prior conventional Fulvestrant formulations and available drug delivery systems novel complex formula of Fulvestrant or derivatives thereof and complexation agents and pharmaceutically acceptable excipients characterized by increased apparent solubility, instantaneous dissolution, increased permeability, which makes the compound orally available making oral administration a possible alternative of the currently used intramuscular formula, Faslodex.

### DESCRIPTION OF THE INVENTION

Disclosed herein is a stable complex comprising as active compound chosen from Fulvestrant, its salts or derivatives thereof; and at least one complexation agent chosen from polyvinylcaprolactam-polyvinyl acetate-polyethylene-glycol graft copolymers; poloxamers; polyvinylpyrrolidone; copolymers of vinylpyrrolidone and vinyl-acetate; and poly(maleic acid-co-methyl-vinyl-ether); said complex characterized in that it possesses at least one of the following properties:
a) is instantaneously redispersable in physiological relevant media
b) is stable in solid form and in colloid solution and/or dispersion;
c) has an apparent solubility in water of at least 1 mg/mL;
d) shows X-ray amorphous character in the solid form;
e) has a PAMPA permeability of at least 0.6*10⁻⁶ cm/s when dispersed in FaSSIF or FeSSIF biorelevant media, which does not decrease in time at least for 1 month; and
f) is characterized by infrared (ATR) spectrum having main/characteristic absorption peaks at least at 1412 cm⁻¹, 1197 cm⁻¹ and 1105 cm⁻¹; and a lack of 1611 cm⁻¹ and 1504 cm⁻¹ characteristic absorption peaks.

The invention is a complex formula having increased apparent solubility and permeability which makes the compound orally available making oral administration a possible alternative of the currently used intramuscular formula, Faslodex.

We have found that only the selected combinations of complexation agents and pharmaceutically acceptable excipients disclosed in the present invention result in a stable complex formulae having improved physicochemical characteristics and enhanced biological performance.

The expression Fulvestrant is generally used for Fulvestrant, or salts or its derivatives.

In an embodiment, said complexation agent is chosen from polyethylene glycol glycerides composed of mono-, di- and triglycerides and mono- and diesters of polyethylene glycol (e.g.; Gelucire 44/14, Gelucire 50/13), hydroxypropylcellulose (e.g; Klucell EF, Klucell LF), poloxamers (copolymers of ethylene oxide and propylene oxide blocks) (e.g; Lutrol F127), vinylpyrrolidone/vinyl acetate copolymer (e.g.; Luviskol VA64), Polyethylene glycol (e.g; PEG2000, PEG6000), poly(2-ethyl-2-oxazoline) (e.g; PEOX50, PEOX500), polyvinylpyrrolidone (e.g; Plasdone K-12, PVP 40, PVP K90, PVP 10), block copolymers based on ethylene oxide and propylene oxide (e.g; Pluronic PE10500, Pluronic PE6800, Pluronic F108), poly(maleic acid/methyl vinyl ether) (PMAMVE), (polyvinyl caprolactam-polyvinyl acetate-polyethylene glycol graft copolymer (e.g; Soluplus), polyoxyl 15 hydroxystearate (e.g; Solutol HS15), ethylene oxide/propylene oxide block copolymer (e.g.; Tetronic 1107), and d-alpha tocopheryl polyethylene glycol 1000 succinate (TPGS).

In an embodiment, said complexation agent is a poloxamer.

In an embodiment, said poloxamer is Poloxamer 407 (Lutrol F127).

In an embodiment, said complex further comprises at least one pharmaceutically acceptable excipient selected from the group of sodium-lauryl-sulfate and sodium-acetate.

In an embodiment, said pharmaceutically acceptable excipient is sodium acetate.

In an embodiment, said complex has a controlled particle size in the range between 50 nm and 600 nm.

In an embodiment, said particle size is between 50 nm and 200 nm.

In an embodiment, said complex has an apparent solubility in water of at least 1.2 mg/mL.

In an embodiment, said complex has an apparent solubility in water of at least 1.4 mg/mL.

In an embodiment, said complex further comprises one or more additional active agents.

In an embodiment, said additional active agent is chosen from agents useful for the treatment hormone receptor positive metastatic breast cancer in postmenopausal women with disease progression following anti-estrogen therapy.

In an embodiment, said additional active agent is chosen from tamoxifen, letrozole, anastrozole, and combinations thereof.

In an embodiment, said complex is orally available.

In an embodiment, said complex possesses at least two of the properties described in a)-f).

In an embodiment, said complex possesses at least three of the properties described in a)-f).

In an embodiment, said complex has an increased dissolution rate.

Further disclosed herein is a stable complex comprising an active compound selected from the group of Fulvestrant, its salt, or derivatives thereof; at least one complexation agent chosen from polyvinylcaprolactam-polyvinyl acetate-polyethylene-glycol graft copolymers; poloxamers; polyvinylpyrrolidone; copolymers of vinylpyrrolidone and vinyl-acetate; and poly(maleic acid-co-methyl-vinyl-ether); and at least one pharmaceutically acceptable excipient chosen from sodium-lauryl-sulfate and sodium-acetate; wherein said complex obtained via a mixing process.

In an embodiment, said complexation agent is a poloxamer.

In an embodiment, said poloxamer is Poloxamer 407 (Lutrol F127).

In an embodiment, said pharmaceutically acceptable excipient is sodium acetate.

In an embodiment, said complex is obtained via a continuous flow mixing process.

In an embodiment, a complex comprises a complexation agent which is a poloxamer and a pharmaceutically acceptable excipient which is sodium-acetate, in a total amount ranging from about 1.0 weight % to about 95.0 weight % based on the total weight of the complex.

In an embodiment, said complexation agent which is a poloxamer and pharmaceutically acceptable excipient which is sodium-acetate comprise 50 weight % to about 95 weight % of the total weight of the complex.

Further disclosed herein is a process for the preparation of the complex, comprising the steps of mixing a solution of Fulvestrant, its salt, or derivatives thereof, and at least one complexation agent chosen from polyvinylcaprolactam-polyvinyl acetate-polyethylene-glycol graft copolymers; poloxamers; polyvinylpyrrolidone; copolymers of vinylpyrrolidone and vinyl-acetate; and poly(maleic acid-co-methyl-vinyl-ether) in a pharmaceutically acceptable solvent with an aqueous solution containing at least one pharmaceutically acceptable excipient chosen from sodium-lauryl-sulfate and sodium-acetate.

In an embodiment, said process is performed in a continuous flow instrument.

In an embodiment, said continuous flow instrument is a microfluidic flow instrument.

In an embodiment, said pharmaceutically acceptable solvent is chosen from methanol, ethanol, isopropanol, n-propanol, acetone, acetonitrile, dimethyl-sulfoxide, tetrahydrofuran, or combinations thereof.

In an embodiment, said pharmaceutically acceptable solvent is n-propanol.

In an embodiment, said pharmaceutically acceptable solvent and said aqueous solvent are miscible with each other.

In an embodiment, said aqueous solvent comprises 0.1 to 99.9% weight of the final solution.

In an embodiment, said aqueous solvent comprises 50 to 90% weight of the final solution.

In an embodiment, said aqueous solvent comprises 50 to 80% weight of the final solution.

In an embodiment, said aqueous solvent comprises 50 to 70% weight of the final solution.

In an embodiment, said aqueous solvent comprises 50 to 60% weight of the final solution.

In an embodiment, said aqueous solvent comprises 45 to 55% weight of the final solution.

In an embodiment, said aqueous solvent comprises 50% weight of the final solution.

In an embodiment, a pharmaceutical composition comprising the complex together with pharmaceutically acceptable carrier.

In an embodiment, said composition is suitable for oral, pulmonary, rectal, colonic, parenteral, intracisternal, intravaginal, intraperitoneal, ocular, otic, local, buccal, nasal, or topical administration.

In an embodiment, said composition is suitable for oral administration.

In an embodiment, said complex is for use in the manufacture of a medicament for the treatment of hormone receptor positive metastatic breast cancer in postmenopausal women with disease progression following anti-estrogen therapy.

In an embodiment, said complex is used for the treatment of hormone receptor positive metastatic breast cancer in postmenopausal women with disease progression following anti-estrogen therapy.

In an embodiment, a method of treatment of hormone receptor positive metastatic breast cancer in postmenopausal women with disease progression following anti-estrogen therapy comprises administration of a therapeutically effective amount of a complex or a pharmaceutical composition as described herein.

In an embodiment, a method for reducing the therapeutically effective dosage of Fulvestrant compared to intramuscular injection comprises oral administration of a pharmaceutical composition as described herein.

Further disclosed herein is a stable complex comprising

a. 10-40% by weight of Fulvestrant, its salt, or derivatives thereof;
b. 20-80% by weight of a poloxamer; and
c. 5-50% by weight of sodium acetate
wherein said complex has a controlled particle size in the range between 50 nm and 600 nm; and
wherein said complex is not obtained via a milling process, high pressure homogenization process, encapsulation process and solid dispersion processes.

In an embodiment, said particle size is between 50 nm and 200 nm.

In an embodiment, said poloxamer is Poloxamer 407 (Lutrol F127).

In an embodiment, said complex shows reduced fed/fasted effect based on in vivo studies.

In an embodiment, said complex shows significantly improved exposure, earlier tₘₐₓ, higher Cₘₐₓ which will allow the oral administration and reduction of the dose.

In an embodiment, said complex has a faster onset of action compared to the existing intramuscular injection formulations.

In an embodiment, said complex is instantaneously redispersable in physiological relevant media.

In an embodiment, said complex is stable in solid form and in colloid solution and/or dispersion.

In an embodiment, said complex has apparent solubility in water of at least 1 mg/mL.

In an embodiment, said complex shows X-ray amorphous character in the solid form.

In an embodiment, said complex has a PAMPA permeability of at least 0.6*10⁻⁶ cm/s when dispersed in FaSSIF or FeSSIF biorelevant media, which does not decrease in time at least for 1 month.

In an embodiment, said complex has a PAMPA permeability of at least 0.4*10⁻⁶ cm/s when dispersed in FaSSIF or FeSSIF biorelevant media, which does not decrease in time at least for 1 month.

In an embodiment, said complex has a PAMPA permeability of at least 0.2*10⁻⁶ cm/s when dispersed in FaSSIF or FeSSIF biorelevant media, which does not decrease in time at least for 1 month.

In an embodiment, said complex is characterized by infrared (ATR) spectrum having main/characteristic absorption peaks at least at 1412 cm⁻¹, 1197 cm⁻¹ and 1105 cm⁻¹; and a lack of 1611 cm⁻¹ and 1504 cm⁻¹ characteristic absorption peaks.

In an embodiment, said complex is further characterized by infrared (ATR) spectrum having main/characteristic absorption peaks at 1577 cm⁻¹ 1467 cm⁻¹, 1359 cm⁻¹, 1343 cm⁻¹, 1281 cm⁻¹, 1242 cm⁻¹, 1146 cm⁻¹, 1060 cm⁻¹, 1012 cm⁻¹, 963 cm⁻¹, 924 cm⁻¹, 842 cm⁻¹, 647 cm⁻¹ and 619 cm⁻¹.

The complexation agents and pharmaceutically acceptable excipients of the Fulvestrant complex formulae of the invention are selected from the group of pharmaceutically acceptable nonionic, anionic, cationic, ionic polymers, surfactants and other types of excipients. The complexation agents themselves or together with the pharmaceutically accepted excipients have the function to form a complex structure with an active pharmaceutical ingredient through non-covalent secondary interactions. The secondary interactions can form through electrostatic interactions such as ionic interactions, H-bonding, dipole-dipole interactions, dipole-induced dipole interactions, London dispersion forces, π-π interactions, and hydrophobic interactions. The complexation agents, pharmaceutically accepted excipients and active ingredients are selected from the group of complexation agents, pharmaceutically accepted excipients and active ingredients which are able to form such complex structures through non-covalent secondary interactions.

In some embodiments, the compositions may additionally include one or more pharmaceutically acceptable excipients, auxiliary materials, carriers, active agents or combinations thereof. In some embodiments, active agents may include agents useful for the treatment hormone receptor positive metastatic breast cancer in postmenopausal women with disease progression following anti-estrogen therapy.

Another aspect of the invention is the complex formulae of the Fulvestrant with complexation agents and pharmaceutically acceptable excipients in which the complexation agents and pharmaceutically acceptable excipients preferably are associated or interacted with the Fulvestrant, such as the results of a mixing process or a continuous flow mixing process. In some embodiment, the structure of the complex Fulvestrant formula is different from the core-shell type milled particle, precipitated encapsulated particles, micelles and solid dispersions.

The pharmaceutical composition of the invention can be formulated: (a) for administration selected from the group consisting of oral, pulmonary, rectal, colonic, parenteral, intracisternal, intravaginal, intraperitoneal, ocular, otic, local, buccal, nasal, and topical administration; (b) into a dosage form selected from the group consisting of liquid dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and (c).

The compositions can be formulated by adding different types of excipients for oral administration in solid, liquid, local (powders, ointments or drops), or topical administration, and the like.

The compositions can be formulated by adding different types of pharmaceutically acceptable excipients for oral administration in solid, liquid, local (powders, ointments or drops), or topical administration, and the like.

In an embodiment, the dosage form of the invention is a solid dosage form, although any pharmaceutically acceptable dosage form can be utilized.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, microcrystalline cellulose and silicic acid; (c) binders, such as cellulose derivatives, alginates, gelatin, polyvinylpyrrolidone, sucrose and acacia; (d) humectants, such as glycerol; (e) disintegrating agents, such as crospovidon, sodium starch glycolate, effervescent compositions, croscarmellose sodium, calcium carbonate, potato or tapioca starch, alginic acid, certain complex silicates and sodium carbonate; (f) solution retarders, such as acrylates, cellulose derivatives, paraffin; (g) absorption accelerators, such as quaternary ammonium compounds; (h) wetting agents, such as polysorbates, cetyl alcohol and glycerol monostearate; (i) adsorbents, such as kaolin and bentonite; and j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Besides such inert diluents, the composition can also include adjuvants, such as wetting agents, emulsifying and suspending agents, sweetening, flavoring, and perfuming agents.

Advantages of the complex Fulvestrant formulae of the invention include, but are not limited to (1) physical and chemical stability, (2) instantaneous redispersibility, (3) stability in colloid solution or dispersion in the therapeutic time window, (4) increased apparent solubility compared to the conventional Fulvestrant formulation, (5) increased permeability, (6) oral bioavailability, (7) decreased fed/fasted effect and (8) good processability.

Beneficial features of the present invention are as follows: the good/instantaneous redispersibility of solid complex formulae of Fulvestrant in water, biologically relevant media, e.g.; physiological saline solution, pH=2.5HCl solution, FessiF and FassiF media and gastro intestinal fluids and adequate stability in colloid solutions and/or dispersion in the therapeutic time window.

In an embodiment, the complex Fulvestrant formulae of the present invention has increased apparent solubility and permeability. In some embodiments, the apparent solubility and permeability of the complex Fulvestrant formulae is at least 1 mg/mL and 0.6*10⁻⁶ cm/s, respectively.

In another embodiment, the complex Fulvestrant formulae of the present invention has an enhanced pharmacokinetic performance. The complex Fulvestrant is orally available making oral administration a possible alternative of the currently used intramuscular formula, Faslodex.

### BRIEF DESCRIPTION OF THE DRAWINGS

FIG. 1. shows comparative PAMPA assays of complex Fulvestrant formulations comprising different complexation agents and pharmaceutically acceptable excipients.

FIG. 2. shows the effect of the ratios of complexation agents and pharmaceutically acceptable excipients on the material characteristics of complex. Fulvestrant formulations: A: Lutrol F127:Fulvestant ratio=0.5:1, B: Lutrol F127:Fulvestant ratio=1:1 and C: Lutrol F127:Fulvestant ratio=2:1.

FIG. 3. shows the particle size distribution of the as-synthesized colloid solution and redispersed solid complex of the selected formula.

FIG. 4. shows comparative dissolution tests of complex Fulvestrant and physical mixture of Fulvestrant, Lutrol F127 and Sodium acetate.

FIG. 5. shows comparative PAMPA assays of complex Fulvestrant formula and unformulated compound.

FIG. 6. shows the stability of the colloid solution in simulated fasted and fed state.

FIG. 7. shows the stability of the solid form detected as the PAMPA permeability measured after redispersion in distilled water after storage at different conditions.

FIG. 8. shows SEM photo of complex Fulvestrant (A) and placebo sample (B).

FIG. 9. shows ATR spectra of crystalline Fulvestrant (A), amorphous Fulvestrant (B), complex Fulvestrant (C), placebo sample (D), Poloxamer 407 (Lutrol F127) (E) and Sodium acetate (F).

FIG. 10. shows powder X-ray diffractograms of crystalline Fulvestrant and complex Fulvestrant formulation.

FIG. 11. shows the plasma concentration following the oral administration of the complex Fulvestrant at 10 mg/kg dose to rats in the fasted condition (n=3).

FIG. 12. shows the plasma Fulvestrant concentrations following the oral administration of 20 mg complex Fulvestrant to beagle dogs (11-13 kg, n=4) in the fasted and in the fed conditions.

### EXAMPLES

Several complexation agents and pharmaceutically acceptable excipients and their combinations were tested in order to select the formulae having instantaneous redispersibility as shown in Error! Reference source not found. One of the examples that displayed an acceptable level of redispersibility was selected for further analysis.

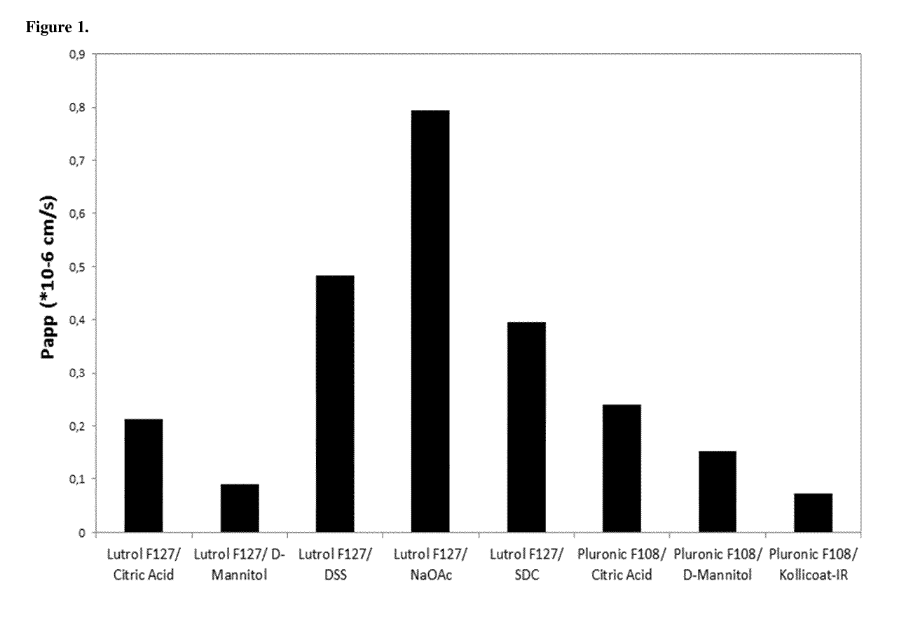

PAMPA permeability of the selected formulations was measured in order to select the complex Fulvestrant formulation having the best in vitro performance (FIG. 1) Error! Reference source not found. PAMPA permeability measurements were performed as described by M. Kansi et al. (Journal of medicinal chemistry, 41, (1998) pp 1007) with modifications based on S. Bendels et al (Pharmaceutical research, 23 (2006) pp 2525). Permeability was measured in a 96-well plate assay across an artificial membrane composed of dodecane with 20% soy lecithin supported by a PVDF membrane (Millipore, USA). The receiver compartment was phosphate buffered saline (pH 7.0) supplemented with 1% sodium dodecyl sulfate. The assay was performed at room temperature; incubation time was 1-24 hours. The concentration in the receiver compartment was determined by UV-VIS spectrophotometry (Thermo Scientific Genesys S10).

Lutrol F127 and Sodium acetate were selected as the complexation agent and pharmaceutically acceptable excipient, respectively, to form complex Fulvestrant formulation having improved material characteristics.

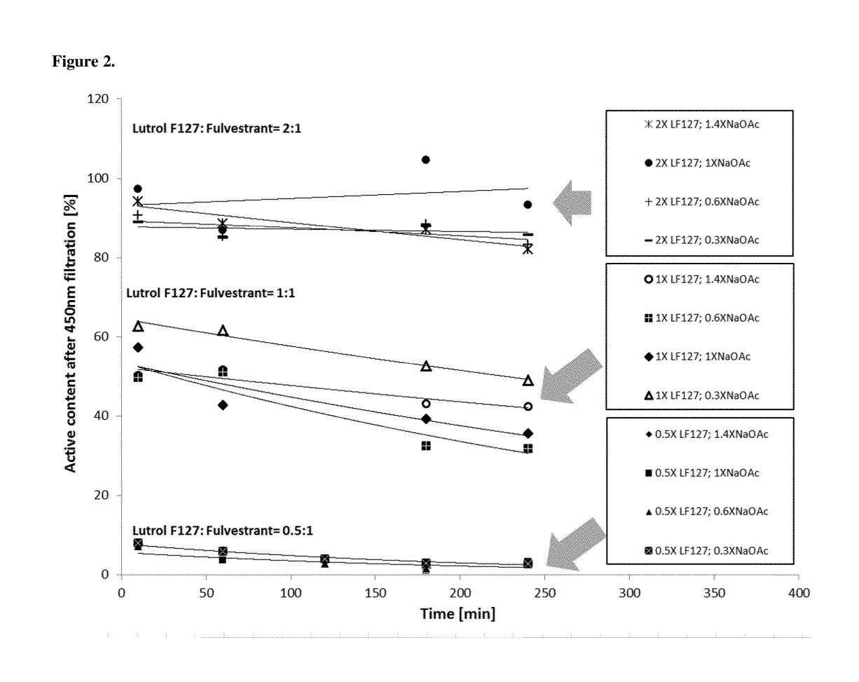

The ratio of the selected complexation agents and pharmaceutically acceptable excipients (Lutrol F127 and Sodium acetate) was optimized. Solid complexes of Fulvestrant were prepared by using different ratios of complexation agents and pharmaceutically acceptable excipients. Lutrol F127:Fulvestrant ratio was kept at 0.5:1, 1:1 and 2:1, while the Sodium acetate ratio in the composition was varied. The solid samples were redispersed in distillated water at 0.4 mg/mL Fulvestrant equivalent concentration. The Fulvestrant contents of the redispersed solutions after filtration (FIG. 2) and PAMPA permeability (Table 2) were used to determine the optimal ratio of complexation agents and pharmaceutically acceptable excipients in the composition (25 weight % Fulvestrant, 50 weight % Lutrol F127 and 25% weight % Sodium acetate) of the complex Fulvestrant of the present invention.

TABLE 2

| | |
|---|---|
| Composition | PAMPA permeability (*10-6 cm/s) |

Lutrol F127:Fulvestrant = 0.5:1

| | |
|---|---|
| 1.4 × Sodium acetate | 0.0000 |
| 1 × Sodium acetate | 0.0000 |
| 0.6 × Sodium acetate | 0.0000 |
| 0.3 × Sodium acetate | 0.0000 |
| 0 | 0.0000 |

Lutrol F127:Fulvestrant = 1:1

| | |
|---|---|
| 1.4 × Sodium acetate | 0.0000 |
| 1 × Sodium acetate | 0.0000 |
| 0.6 × Sodium acetate | 0.0000 |
| 0.3 × Sodium acetate | 0.0276 |
| 0 | 0.0000 |

Lutrol F127:Fulvestrant = 2:1

| | |
|---|---|
| 1.4 × Sodium acetate | 0.2175 |
| 1 × Sodium acetate | 0.5116 |
| 0.6 × Sodium acetate | 0.2717 |
| 0.3 × Sodium acetate | 0.1939 |
| 0 | 0.1433 |

A colloid solution of Fulvestrant complex formula with the optimal ratio of complexation agents and pharmaceutically acceptable excipients of the present invention was prepared by continuous flow mixing in a flow instrument. As a starting solution, 200 mg Fulvestrant and 400 mg Poloxamer 407 (Lutrol F127) dissolved in 100 mL n-propanol was used. The prepared solution was passed into the instrument with 2 mL/min flow rate. Meanwhile, aqueous solvent containing 250 mg sodium-acetate in 500 mL water was passed into the instrument with 8 mL/min flow rate, where Fulvestrant formed complex Fulvestrant composition. The colloid solution of the complex Fulvestrant is continuously produced at atmospheric pressure. The produced colloid solution was frozen on dry-ice and then it was lyophilized using a freeze drier equipped with −110° C. ice condenser, with a vacuum pump. For the process monitoring particle size of the redispersed complex Fulvestrant formula was used. Particle size of the colloid solutions prepared with different flow rates and the particle size of the reconstituted solid complex Fulvestrant formulae are seen in Table 3.

TABLE 3

| Flow rate of organic | | |
|---|---|---|
| solution containing | Flow rate of Aqueous | Redsipersed particle size |
| Fulvestrant (mL/min) | solution (mL/min) | dmean (nm) |
| 1 | 4 | 188 |
| 2 | 8 | 186 |
| 4 | 16 | 186 |
| 8 | 32 | 177 |

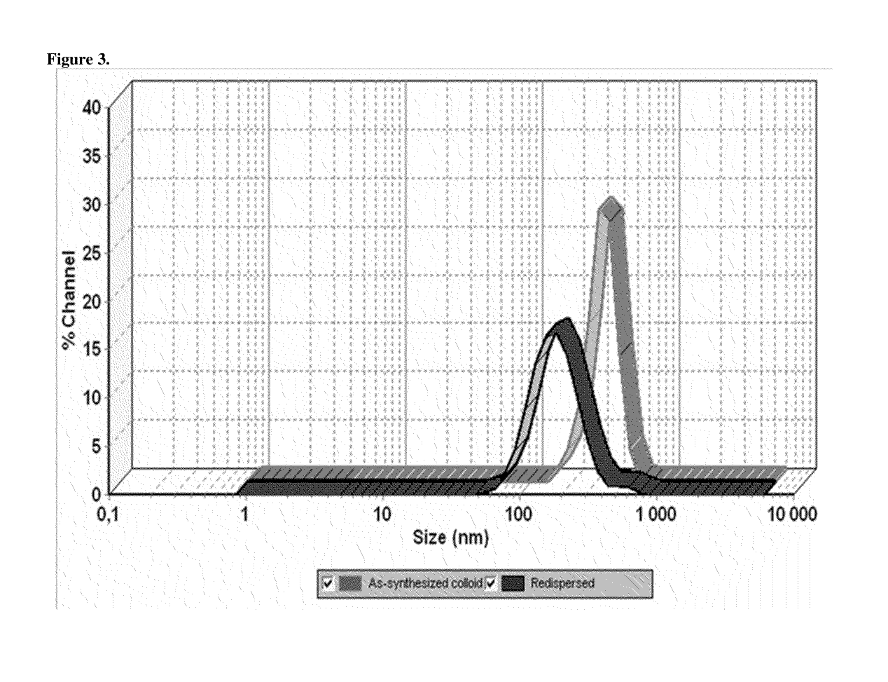

The stability of the reconstituted solid complex Fulvestrant formula was monitored. Based on the physical appearance and stability of the reconstituted solid complex Fulvestrant formula, the best composition was selected for analytical investigations (FIG. 3).

TABLE 3

| Flow rate of organic | | |
|---|---|---|
| solution containing | Flow rate of Aqueous | Redsipersed particle size |
| Fulvestrant (mL/min) | solution (mL/min) | dₘₑₐₙ (nm) |
| 1 | 4 | 188 |
| 2 | 8 | 186 |
| 4 | 16 | 186 |
| 8 | 32 | 177 |

In order to make the production process industrially feasible, process intensification was performed by increasing the concentrations of the starting soluting. A colloid solution of Fulvestrant complex formula of the present invention was prepared by continuous flow mixing in a flow instrument using the intensified process parameters. As a starting solution, 1400 mg Fulvestrant and 2800 mg Poloxamer 407 (Lutrol F127) dissolved in 100 mL n-propanol was used. The prepared solution was passed into the instrument with 10 mL/min flow rate. Meanwhile, aqueous solvent containing 1750 mg sodium-acetate in 500 mL water was passed into the instrument with 40 mL/min flow rate, where Fulvestrant formed complex Fulvestrant composition.

Different flow rate ratios were tested to determine the optimal manufacturing condition. The appearance of the produced solvent mixture and the Fulvestrant content of the solution mixture after filtration were used to determine the optimal parameter of the production. Table 4 summarizes the results.

Based on the results flow rate ratio of 5 mL/min:20 mL/min was selected for further optimization.

TABLE 4
Effect of the flow rate ratio on the appearance and active content of the
solvent mixture after filtration

| Flow rate ratios (organic phase:aqueous phase) | | | |
|---|---|---|---|
| (mL/min:mL/min) | | | |
| 5:5 | 5:10 | 5:15 | 5:20 |

API content of the solution mixture after filtration through 450 nm
PTFE filter

| | | | | |
|---|---|---|---|---|
| C_{Fulvestrant} (mg/ml) | 1.8 | 2.4 | 1.7 | 2.8 |
| Appearance | transparent | opalescent | opalescento | palescent colloid |

With the optimized flow rate ratio (5:20), solvent mixture containing novel Fulvestrant formulation was prepared and solid formulated using freeze-drying method. The stability of the freeze-dried powder was tested after one week storage at 4° C. The samples were reconstituted using purified water. The physical stability of obtained opalescent solution was also monitored in time by the determination of the Fulvestrant content of the colloid solution after filtration. The results are summarized in Table 5.

TABLE 5
Fulvestrant content of the filtrate at different time points

| | Right after the production | After one week storage |
|---|---|---|
| Time | C_{Fulvestrant} (mg/ml) | C_{Fulvestrant} (mg/ml) |
| t = 0 | 0.248 | 0.226 |
| t = 0.5 h | 0.239 | 0.200 |
| t = 1 h | 0.234 | 0.196 |
| t = 2 h | 0.225 | 0.180 |
| t = 4 h | 0.213 | 0.175 |

The results showed that the Fulvestrant content of the filtrate right after the production was identical to Fulvestrant content of the filtrate after one week storage within the experimental accuracy. The Fulvestrant content of the filtrates slightly decreases in time; however it does not have effect on the appearance and redispersibility of the freeze-dried powder.

### Effect of the Process Intensification (Process Scale-Up) on the In Vitro Properties

Process intensification was also performed in order to increase the efficiency of the production. The flow rate ratios were increased from 5:20 up to 10:40. The produced solvent mixtures were solid formulated using freeze-drying method. The stability of the freeze-dried powders was tested after one week storage at 5±3° C. The samples were reconstituted using purified water. The physical stability of obtained opalescent solution was also monitored in time by the determination of the Fulvestrant content of the colloid solution after filtration. The results are summarized in Table 6.

TABLE 6
Effect of the process intensification on the redispersibility
of novel Fulvestrant formulation

| | C_{Fulvestrant} (mg/ml) | | | | | |
|---|---|---|---|---|---|---|
| | Flow rate ratio (mL/min:mL/min) | | | | | |
| | 5:20 | 7.5:30 | 10:40 | 5:20 | 7.5:30 | 10:40 |
| Time | Right after the production | | | After one week storage | | |
| t = 0 | 0.248 | 0.222 | 0.235 | 0.226 | 0.234 | 0.236 |
| t = 0.5 h | 0.239 | 0.208 | 0.222 | 0.200 | 0.216 | 0.229 |
| t = 1 h | 0.234 | 0.191 | 0.218 | 0.196 | 0.211 | 0.229 |
| t = 2 h | 0.225 | 0.170 | 0.225 | 0.180 | 0.202 | 0.229 |
| t = 4 h | 0.213 | 0.159 | 0.179 | 0.175 | 0.162 | 0.204 |

The results showed that the intensification of the production did not modify the determined material characteristics of the produced novel Fulvestrant formulations. The Fulvestrant contents of the filtrates of the reconstituted novel formulations prepared with different flow rates were almost identical within the experimental accuracy. The results also showed that the Fulvestrant content of the filtrate right after the production was almost identical to Fulvestrant content of the filtrate after one week storage. The Fulvestrant content of the filtrates slightly decreases in time; however it does not have influence on the redispersibility and stability of the freeze-dried powder. Based on the results, the flow rate ratio can be increased up to 10 mL/min:40 mL/min without losing the redispersibility and stability characteristics of the novel formulation.

### Comparative Solubility Tests

The apparent solubility of complex Fulvestrant formula and unformulated compounds was measured by UV-VIS spectroscopy at room temperature. The samples were dispersed in distillated water and the resulting dispersions were filtered by 100 nm disposable syringe filter. The active content in the filtrate was measured by UV-Vis spectrophotometry and the solubility was calculated. The filtrate may contain Fulvestrant complex particles which could not be filtrated out using 100 nm pore size filter.

Solubility of complex Fulvestrant formula and unformulated compound was 1.43 mg/mL and <0.03 mg/mL, respectively.

### Comparative Dissolution Tests

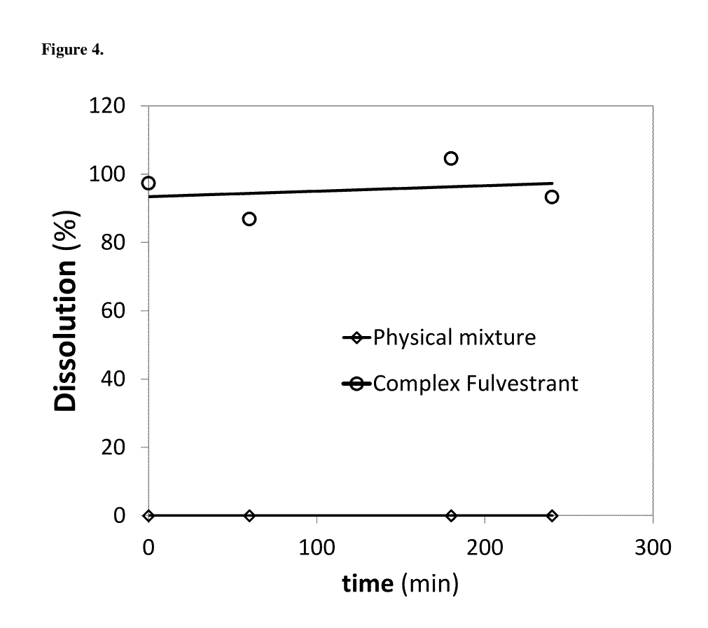

Comparative dissolution tests were performed by redispersing the complex Fulvestrant formulation and physical mixture of Fulvestrant, Lutrol F127 and Sodium acetate in purified water at 0.25 mg/mL concentrations. The dissolved amount was measured with UV-VIS spectrophotometry after filtration with 0.45 μm pore size filter at different time points. Dissolution of Fulvestrant from the complex formulation was instantaneous, while the dissolution of Fulvestrant from the physical mixture could not be detected (FIG. 4).

### Comparative In Vitro PAMPA Assays

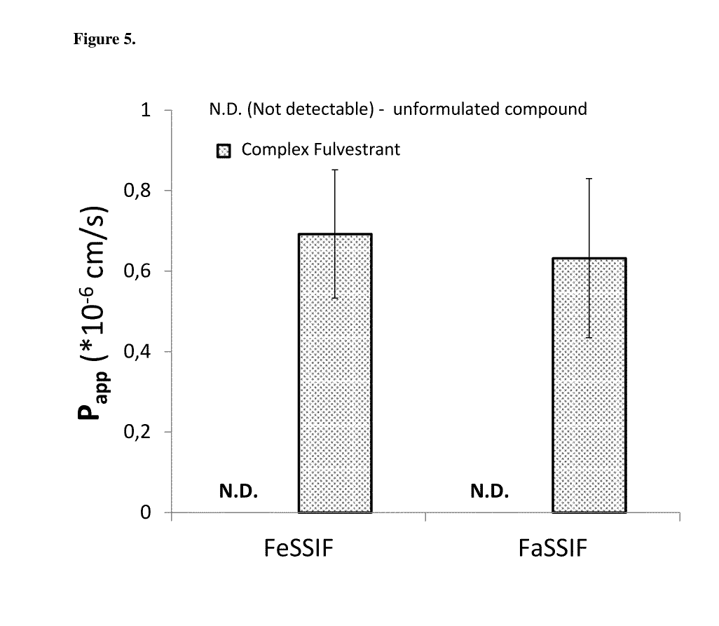

PAMPA permeability of complex Fulvestrant formula was above 0.6*10⁻⁶ cm/s in both the fed and fasted state simulating conditions, while it was not detectable for the unformulated compound, see FIG. 5.

### Stability of the Colloid Solution in the GI Tract

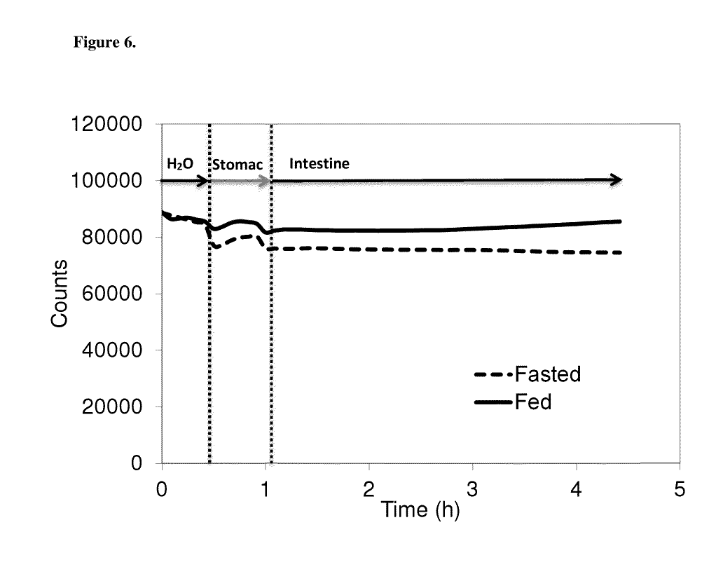

A simulated passage through the GI tract was performed in order to detect any instability of the colloid solution at pH values and bile acid concentrations representative of the GI tract in the fasted and in the fed conditions. No significant change in light scattering of the colloid solution was observed in the simulation indicating that the formula will be stable under these conditions in the time window of the absorption process in both the fasted and in the fed conditions (FIG. 6).

### Stability of the Solid Form

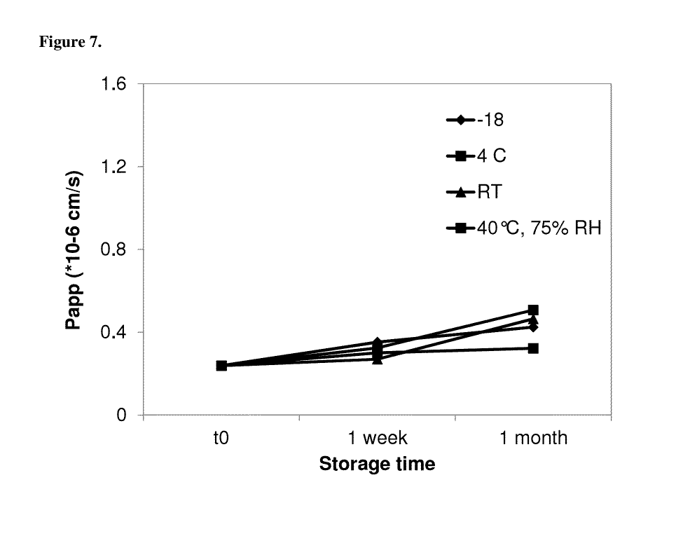

The stability testing of the solid form is ongoing. PAMPA permeability of the solid is measured after storage at different conditions. 1 month storage at −18° C., 4° C., RT or 40° C. 75% relative humidity showed no significant decrease in the measured PAMPA permeability under any of the conditions tested (FIG. 7).

### Structural Analysis

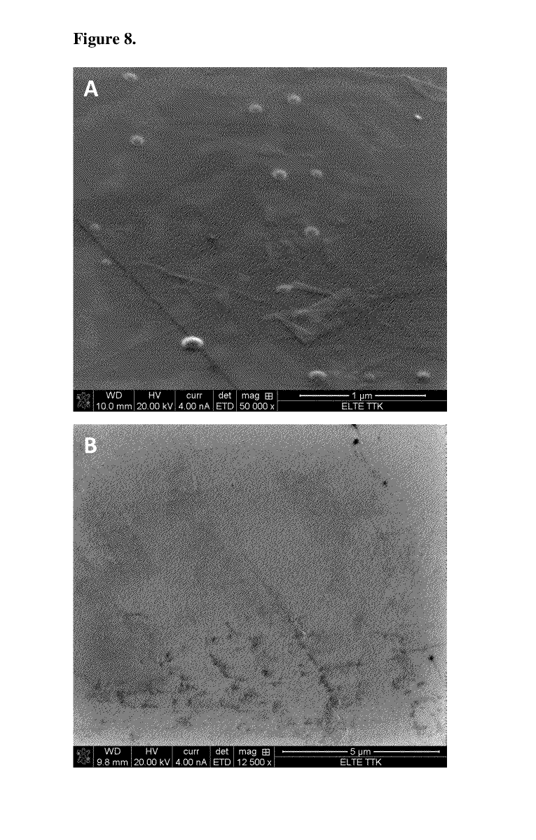

Morphology of complex Fulvestrant was investigated using FEI Quanta 3D scanning electron microscope. The morphology of the complex of the present invention was compared to the placebo sample, prepared as described above, containing sodium acetate and Lutrol F127 but lacking Fulvestrant. Complex Fulvestrant of the present invention consists of spherical particles (FIG. 8. A). In the lack of the active compound, the complexation agents and pharmaceutically acceptable excipients do not form spherical particles (FIG. 88. B).

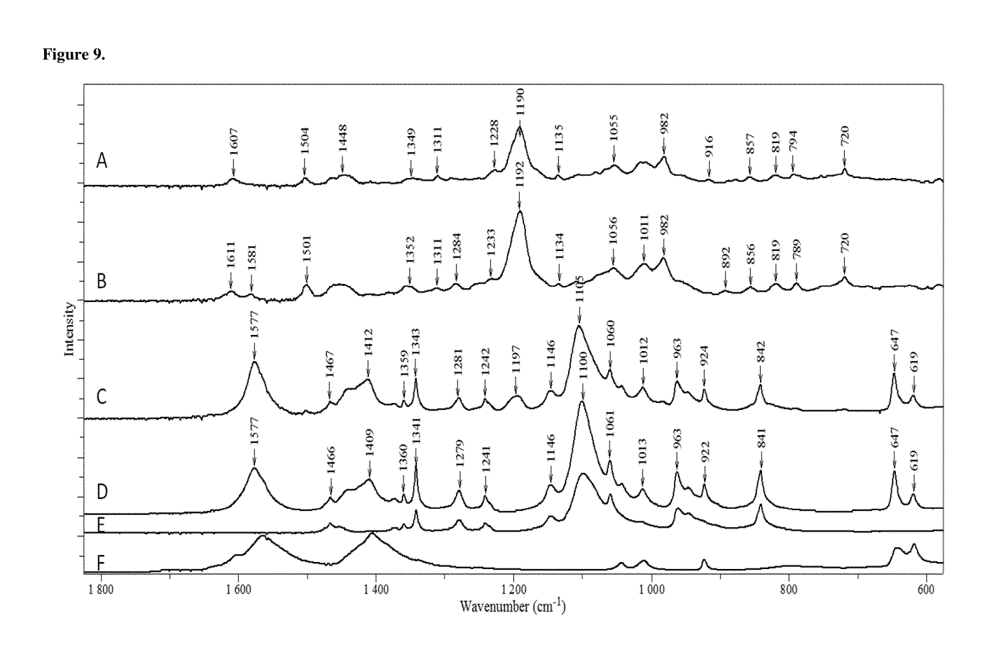

Structural analysis was performed by using Bruker Vertex 70 FT-IR spectrometer with Bruker Platinum diamond ATR unit. Continuous flow mixing of Fulvestrant in the presence of selected complexation agents and pharmaceutically acceptable excipients, such as Poloxamer 407 (Lutrol F127) and sodium acetate resulted in a stable complex of Fulvestrant. In a embodiment the complex or the pharmaceutical composition according to the invention characterized by at least one of the following characteristic Raman or ATR band/peak (FIG. 9):

1577 cm⁻¹, 1467 cm⁻¹, 1412 cm⁻¹ 1359 cm⁻¹, 1343 cm⁻¹, 1281 cm⁻¹, 1242 cm⁻¹, 1197 cm⁻¹, 1146 cm⁻¹, 1105 cm⁻¹, 1060 cm⁻¹, 1012 cm⁻¹, 963 cm⁻¹, 924 cm⁻¹, 842 cm⁻¹, 647 cm⁻¹ and 619 cm⁻¹, preferable at 1412 cm⁻¹, 1197 cm⁻¹ and 1105 cm⁻¹; or the lack of 1607/1611 cm⁻¹ and 1501/1504 cm⁻¹ characteristic absorption peaks.

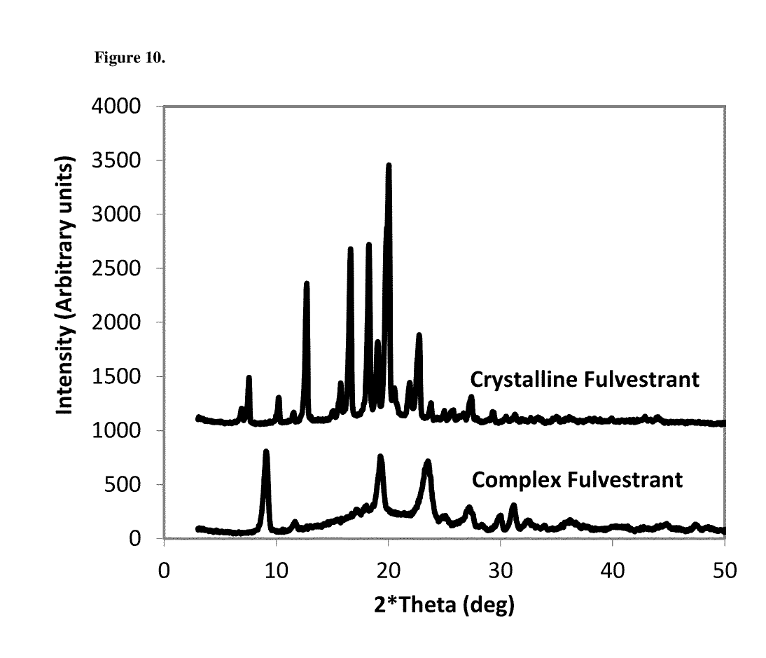

The structure of the complex Fulvestrant of the present invention was investigated by powder X-ray diffraction (XRD) analysis (Philips PW1050/1870 RTG powder-diffractometer). The measurements showed that the complex Fulvestrant composition was XRD amorphous (See in FIG. 10.). Characteristic reflections on the diffractogram of complex Fulvestrant could be attributed to the Sodium acetate in the formulation.

### In-Vivo Pharmacokinetics

### In-Vivo PK Test in Small Animals

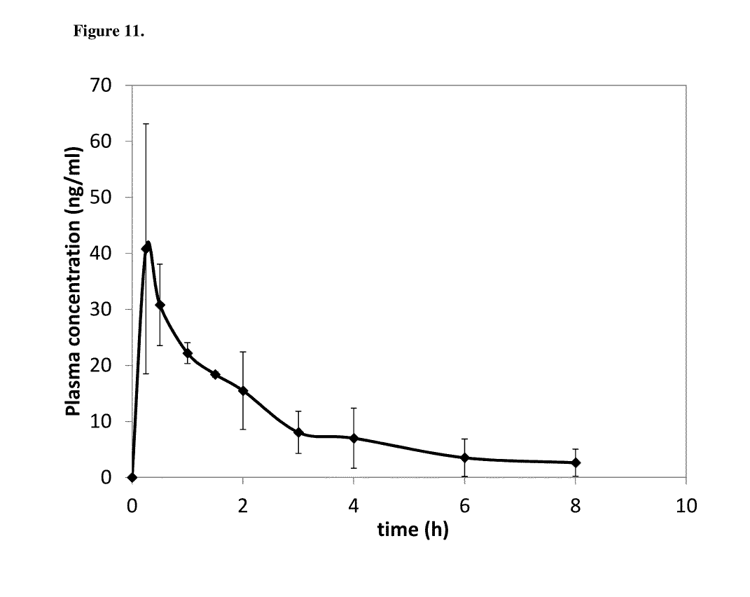

Following oral administration of the complex Fulvestrant formula to fasted rats the highest plasma concentrations were detected at the first time point (15 min) indicating very fast absorption of the active from similar to intravenous administration (FIG. 11). Since no reference formula was available for comparison relative bioavailability cannot be calculated, however, the intravenous like absorption/elimination profile allowed the estimation of pharmacokinetic properties in rats (n=3), see Table 7. The compound exhibits very high volume of distribution, therefore, in order to estimate PK parameters later time points (1.5-8 h) were used. It is interesting to note that at this dose level Cₘₐₓ values exceeded the human therapeutic plasma concentrations by around 5-10-fold.

TABLE 7

| PK parameter | Value |
|---|---|
| AUC (h*ng/ml) | 81.9 |
| t_{1/2} (h) | 1.54 |
| Cₘₐₓ (ng/ml) | 40.8 |
| tₘₐₓ (h) | 0.25 |

### In-Vivo PK Test in Large Animals

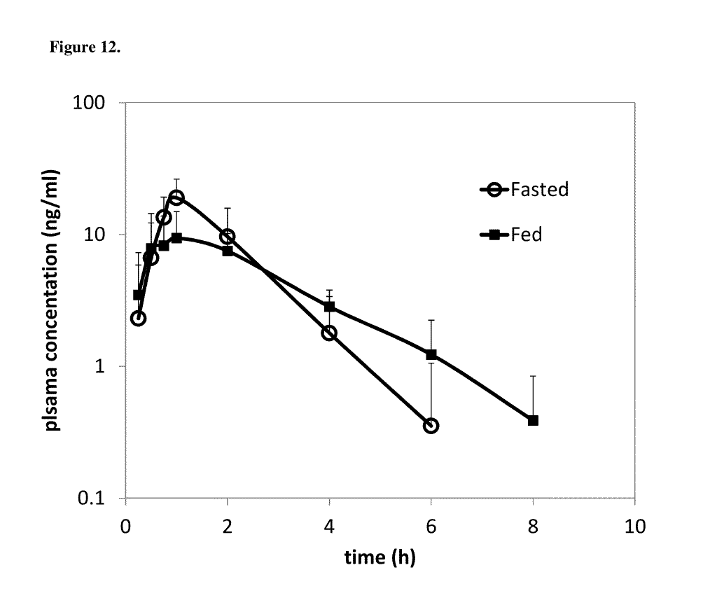

A beagle dog study using a powder in a bottle formula with 20 mg strength was performed in the fasted and in the fed condition (FIG. 12). The formula was administered as a reconstituted colloid solution. The absorption of the API was fast with tₘₐₓ values at 1 hour. Inter-individual differences were small. A slight food effect with lower cₘₐₓ in the fed condition, but essentially the same exposure was found. Clearance (estimated from t_{1/2}) was even higher, than in rats (t_{1/2}=1.54 h) (Table 8).

TABLE 8

| PK parameter | Fasted | Fed |
|---|---|---|
| Cₘₐₓ (ng/ml) | 19.5 ± 7.3 | 11.0 ± 4.8 |
| Tₘₐₓ (h) | 0.93 ± 0.13 | 1.35 ± 0.75 |
| AUC_{0-∞} (ng*h/ml) | 36.2 ± 17.9 | 31.2 ± 9.2 |
| t_{1/2} (h) | 0.86 | 1.51 |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A stable complex comprising as active compound chosen from Fulvestrant, its salts or derivatives thereof; and at least one complexation agent chosen from polyvinylcaprolactam-polyvinyl acetate-polyethylene-glycol graft copolymers; poloxamers; polyvinylpyrrolidone; copolymers of vinylpyrrolidone and vinyl-acetate; and poly(maleic acid-co-methyl-vinyl-ether); said complex characterized in that it possesses at least one of the following properties:
a) is instantaneously redispersable in physiological relevant media
b) is stable in solid form and in colloid solution and/or dispersion;
c) has an apparent solubility in water of at least 1 mg/mL;
d) shows X-ray amorphous character in the solid form;
e) has a PAMPA permeability of at least 0.6*10⁻⁶ cm/s when dispersed in FaSSIF or FeSSIF biorelevant media, which does not decrease in time at least for 1 month; and
f) is characterized by infrared (ATR) spectrum having main/characteristic absorption peaks at least at 1412 cm⁻¹, 1197 cm⁻¹ and 1105 cm⁻¹; and a lack of 1611 cm⁻¹ and 1504 cm⁻¹ characteristic absorption peaks.

2. The complex as recited in claim 1, wherein said complexation agent is chosen from polyethylene glycol glycerides composed of mono-, di- and triglycerides and mono- and diesters of polyethylene glycol (e.g.; Gelucire 44/14, Gelucire 50/13), hydroxypropylcellulose (e.g; Klucell EF, Klucell LF), poloxamers (copolymers of ethylene oxide and propylene oxide blocks) (e.g; Lutrol F127), vinylpyrrolidone/vinyl acetate copolymer (e.g.; Luviskol VA64), Polyethylene glycol (e.g; PEG2000, PEG6000), poly(2-ethyl-2-oxazoline) (e.g; PEOX50, PEOX500), polyvinylpyrrolidone (e.g; Plasdone K-12, PVP 40, PVP K90, PVP 10), block copolymers based on ethylene oxide and propylene oxide (e.g; Pluronic PE10500, Pluronic PE6800, Pluronic F108), poly(maleic acid/methyl vinyl ether) (PMAMVE), (polyvinyl caprolactam-polyvinyl acetate-polyethylene glycol graft copolymer (e.g; Soluplus), polyoxyl 15 hydroxystearate (e.g; Solutol HS15), ethylene oxide/propylene oxide block copolymer (e.g.; Tetronic 1107), and d-alpha tocopheryl polyethylene glycol 1000 succinate (TPGS).

3. The complex as recited in claim 1, wherein said complexation agent is a poloxamer.

4. The complex as recited in claim 1, wherein said poloxamer is Poloxamer 407 (Lutrol F127).

5. The complex as recited in claim 1, wherein said complex further comprises at least one pharmaceutically acceptable excipient selected from the group of sodium-lauryl-sulfate and sodium-acetate.

6. The complex as recited in claim 5, wherein said pharmaceutically acceptable excipient is sodium acetate.

7. The complex as recited in claim 1, wherein said complex has a controlled particle size in the range between 50 nm and 600 nm.

8. The complex as recited in claim 7, wherein said particle size is between 50 nm and 200 nm.

9. The complex as recited in claim 1, wherein said complex possesses at least two of the properties described in a)-f).

10. The complex as recited in claim 1, wherein said complex possesses at least three of the properties described in a)-f).

11. The complex as recited in claim 1, wherein said complex has an increased dissolution rate.

12. A stable complex comprising an active compound selected from the group of Fulvestrant, its salt, or derivatives thereof; at least one complexation agent chosen from polyvinylcaprolactam-polyvinyl acetate-polyethylene-glycol graft copolymers; poloxamers; polyvinylpyrrolidone; copolymers of vinylpyrrolidone and vinyl-acetate; and poly(maleic acid-co-methyl-vinyl-ether); and at least one pharmaceutically acceptable excipient chosen from sodium-lauryl-sulfate and sodium-acetate; wherein said complex obtained via a mixing process.

13. The complex as recited in claim 12, wherein said complexation agent is a poloxamer.

14. The complex as recited in claim 13, wherein said poloxamer is Poloxamer 407 (Lutrol F127).

15. The complex as recited in claim 12, wherein said pharmaceutically acceptable excipient is sodium acetate.

16. The complex as recited in claim 12, wherein said complex is obtained via a continuous flow mixing process.

17. A complex according to claim 1 comprising a complexation agent which is a poloxamer and a pharmaceutically acceptable excipient which is sodium-acetate, in a total amount ranging from about 50.0 weight % to about 95.0 weight % based on the total weight of the complex.

18. A process for the preparation of the complex according to claim 12, comprising the steps of mixing a solution of Fulvestrant, its salt, or derivatives thereof, and at least one complexation agent chosen from polyvinylcaprolactam-polyvinyl acetate-polyethylene-glycol graft copolymers; poloxamers; polyvinylpyrrolidone; copolymers of vinylpyrrolidone and vinyl-acetate; and poly(maleic acid-co-methyl-vinyl-ether) in a pharmaceutically acceptable solvent with an aqueous solution containing at least one pharmaceutically accepted excipient chosen from sodium-lauryl-sulfate and sodium-acetate.

19. The process as recited in claim 18, wherein said process is performed in a continuous flow instrument.

20. The process as recited in claim 19, wherein said continuous flow instrument is a microfluidic flow instrument.

21. The process as recited in claim 18, wherein said pharmaceutically acceptable solvent is chosen from methanol, ethanol, isopropanol, n-propanol, acetone, acetonitrile, dimethyl-sulfoxide, tetrahydrofuran, or combinations thereof.

22. The process as recited in claim 21, wherein said pharmaceutically acceptable solvent is n-propanol.

23. A pharmaceutical composition comprising the complex according to claim 1 together with pharmaceutically acceptable carrier.

24. A pharmaceutical composition comprising the complex according to claim 1 wherein said composition is suitable for oral, pulmonary, rectal, colonic, parenteral, intracisternal, intravaginal, intraperitoneal, ocular, otic, local, buccal, nasal, or topical administration.

25. The pharmaceutical composition comprising the complex according to claim 24, wherein said composition is suitable for oral administration.

26. A method of treatment of hormone receptor positive metastatic breast cancer in postmenopausal women with disease progression following anti-estrogen therapy comprising administration of a therapeutically effective amount of the complex according to claim 1.

27. A method for reducing the therapeutically effective dosage of Fulvestrant compared to intramuscular injection, said method comprising oral administration of a pharmaceutical composition according to claim 23.

28. A stable complex comprising
a) 10-40% by weight of Fulvestrant, its salt, or derivatives thereof;
b) 20-80% by weight of a poloxamer; and
c) 5-50% by weight of sodium acetate
d) wherein said complex has a controlled particle size in the range between 50 nm and 600 nm; and
e) wherein said complex is not obtained via a milling process, high pressure homogenization process, encapsulation process and solid dispersion processes.

29. The complex as recited in claim 28, wherein said particle size is between 50 nm and 200 nm.

30. The complex as recited in claim 29, wherein said poloxamer is Poloxamer 407 (Lutrol F127).
